(19) United States
(12) Patent Application Publication
(10) Pub. No.: US 2016/0051198 A1
(43) Pub. Date: Feb. 25, 2016

(54) SYSTEM FOR MONITORING USER UTILIZING PULSE SIGNAL

(71) Applicant: Industry-Academic Cooperation Foundation, Yonsei University, Seoul (KR)

(72) Inventor: Tae Wook KIM, Seoul (KR)

(21) Appl. No.: 14/831,780

(22) Filed: Aug. 20, 2015

(30) Foreign Application Priority Data
Aug. 20, 2014 (KR) 10-2014-0108129

(51) Int. Cl. A61B 5/00; A61B 5/0245; A61B 5/024

(52) U.S. Cl. CPC A61B 5/6893; A61B 5/02444; A61B 5/0245; A61B 5/02438; A61B 5/7278; A61B 2562/046

(57) ABSTRACT

The present disclosure herein relates to a system for monitoring a user using a pulse signal. An user monitoring system according to an embodiment of the present invention may comprise a transmitter configured to transmit a pulse signal through at least one antenna at different times; and a biosignal measuring device comprising a receiver for receiving and restoring the pulse signal that has passed through a subject through at least one antenna and a processing unit for processing the pulse signal to analyze at least one of a bandwidth, a center frequency or an amplitude of the pulse signal and for measuring a biosignal of the subject on the basis of a result of analysis.

### CROSS-REFERENCE TO RELATED APPLICATIONS

This U.S. non-provisional patent application claims priority under 35 U.S.C. §119 of Korean Patent Application No. 10-2014-0108129, filed on Aug. 20, 2014, the entire contents of which are hereby incorporated by reference.

### BACKGROUND

The present disclosure herein relates to a system for monitoring a user using a pulse signal.

It is a widely used method to contact an electrode to a body of a subject to measure an electrocardiogram in order to detect heart conditions. However, the method of measuring an electrocardiogram using an electrode is a contact-type method that requires contact between a body and an electrode, which may cause inconvenience.

To resolve the issue of inconvenience of the contact-type electrocardiogram measuring method, a contactless electrocardiogram measuring method has been proposed. According to this method, a radio signal is transmitted towards a heart of a subject and then an electrocardiogram is measured using a reflected wave from the heart.

However, since the electrocardiogram is measured on the basis of a change in a distance between a transceiver and a body using a reflected wave, a movement of a subject may cause a serious error.

### SUMMARY

The present disclosure provides a biosignal measuring apparatus for accurately measuring a biosignal such as a heartbeat without being affected by a movement of a subject, and a user monitoring system including the same.

The present disclosure also provides a biosignal measuring apparatus for measuring a biosignal even with low-performance hardware capable of processing signals at a low rate and with low power consumption, and a user monitoring system including the same.

The present disclosure also provides a biosignal measuring apparatus for accurately measuring a biosignal by using only a pulse signal transmitted through a straight path between a transmitter and a receiver with avoidance of interference by a reflected signal transmitted through a multi-path, and a user monitoring system including the same.

Embodiments of the present disclosure provide a user monitoring system comprising: a transmitter configured to transmit a pulse signal through at least one antenna at different times; and a biosignal measuring device comprising a receiver for receiving and restoring the pulse signal that has passed through a subject through at least one antenna and a processing unit for processing the pulse signal to analyze at least one of a bandwidth, a center frequency or an amplitude of the pulse signal and for measuring a biosignal of the subject on the basis of a result of analysis.

In some embodiments, a pulse of the pulse signal may be repeated at a preset pulse repetition period.

In other embodiments, the transmitter may comprise a delay unit configured to provide, to the antenna of the transmitter, a plurality of pulse signals obtained by different delays to the pulse signal.

In even other embodiments, the receiver may comprise: a delay unit configured to apply different delays to a plurality of pulse signals received by the antenna of the receiver; and an adder configured to add the plurality of pulse signals output by the delay unit.

In yet other embodiments, the transmitter and the receiver may be synchronized with each other to share a clock signal.

In further embodiments, the user monitoring system may be provided to a vehicle to monitor the subject in the vehicle.

In still further embodiments, the antenna of the transmitter may be installed in a steering wheel of the vehicle, and the antenna of the receiver may be installed in a back of a driver's seat of the vehicle.

In even further embodiments, the antenna of the transmitter may be installed at a front upper part in the vehicle, and the antenna of the receiver may be installed in a back of a seat of the vehicle.

In yet further embodiments, a plurality of antennas may be such arranged that the antennas are flush with each other and form a shape of a polygon.

In much further embodiments, the user monitoring system may further comprise an antenna disposed at a center of the polygon.

In still much further embodiments, the user monitoring system may further comprise an input device configured to receive information on the seat of the vehicle on which the subject is seated, wherein the transmitter transmits the pulse signal towards the antenna installed in the back of the seat on which the subject is seated.

In even much further embodiments, in the case where the subject is seated on a plurality of seats, the transmitter may transmit the pulse signal towards an antenna installed in a back of each seat on which the subject is seated at different times.

In yet much further embodiments, the user monitoring system may further comprise an input device configured to receive information on a physique of the subject in the vehicle, wherein the receiver comprises a first antenna group installed in a first part of a back of a seat of the vehicle and a second antenna group installed in a second part of the back of the seat, wherein the transmitter transmits the pulse signals towards one of the first and second antenna groups which corresponds to the physique of the subject.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a block diagram schematically illustrating a user monitoring system according to an exemplary embodiment of the inventive concept;

FIG. 2 is a block diagram schematically illustrating a bio signal measuring apparatus according to an exemplary embodiment of the inventive concept;

FIG. 3 is a diagram schematically illustrating a waveform of a time-domain pulse signal used to measure a bio signal of a to-be-measured person, according to an exemplary embodiment of the inventive concept;

FIG. 4 is a diagram schematically illustrating power spectrum of a pulse signal used to measure a bio signal of a to-be-measured person, according to an exemplary embodiment of the inventive concept;

FIG. 5 is a block diagram schematically illustrating a processing unit according to an exemplary embodiment of the inventive concept;

FIGS. 6 and 7 are diagrams schematically illustrating time-domain waveforms and frequency-domain power spectrums after a pulse signal passes through a contracted heart and a dilated heart, according to an exemplary embodiment of the inventive concept;

FIG. 8 is a diagram for describing a method for measuring a heartbeat using a bandwidth of a pulse signal, according to an exemplary embodiment of the inventive concept;

FIG. 9 is a diagram for describing a method for measuring a heartbeat using variations in bandwidth before and after a pulse signal penetrates a heart, according to an exemplary embodiment of the inventive concept;

FIG. 10 is a diagram for describing a method for measuring a heartbeat using a bandwidth and a center frequency of a pulse signal, according to an exemplary embodiment of the inventive concept;

FIG. 11 is a diagram for describing a method for measuring a heartbeat using a bandwidth variation and a center frequency variation of a pulse signal, according to another exemplary embodiment of the inventive concept;

FIG. 12 is a diagram for describing a method for measuring a heartbeat using a bandwidth and amplitude of a pulse signal, according to still another exemplary embodiment of the inventive concept;

FIG. 13 is a diagram for describing a method for measuring a heartbeat using a bandwidth variation and an amplitude variation of a pulse signal, according to still another exemplary embodiment of the inventive concept;

FIG. 14 is a diagram for describing a method for measuring a heartbeat by analyzing a bandwidth using duration of a pulse signal, according to still another exemplary embodiment of the inventive concept;

FIG. 15 is a diagram for describing a method for measuring a heartbeat by analyzing a center frequency using a time difference t between a pulse portion having positive amplitude and a pulse portion having negative amplitude, according to a further exemplary embodiment of the inventive concept;

FIGS. 16 and 17 are diagrams for describing a method for a sampling unit sampling a pulse signal, according to an exemplary embodiment of the inventive concept;

FIG. 18 is a diagram for describing a method in which a sampling unit samples a pulse signal, according to another exemplary embodiment of the inventive concept;

FIG. 19 is a block diagram schematically illustrating a user monitoring system according to another exemplary embodiment of the inventive concept;

FIG. 20 is a diagram for describing a method in which a transmitter transmits pulse signals, according to another exemplary embodiment of the inventive concept;

FIG. 21 is a diagram for describing a method in which a receiver receives a pulse signal, according to another exemplary embodiment of the inventive concept;

FIG. 22 is a diagram for describing an interference signal receiving method of a receiver, according to another exemplary embodiment of the inventive concept;

FIG. 23 is a diagram schematically illustrating arrangements of transmitter antennas and receiver antennas in a vehicle according to an exemplary embodiment of the inventive concept;

FIG. 24 is a diagram schematically illustrating a state where transmitter antennas and receiver antennas are disposed in a vehicle, according to another exemplary embodiment of the inventive concept;

FIGS. 25 to 27 are diagrams schematically illustrating arrangement of antennas according to exemplary embodiments of the inventive concept; and

FIG. 28 is a diagram schematically illustrating arrangement of receiver antennas in a vehicle, according to still another exemplary embodiment of the inventive concept.

### DETAILED DESCRIPTION

According to an embodiment of the inventive concept, a biosignal such as a heartbeat may be accurately measured without being affected by a movement of a subject.

According to an embodiment of the inventive concept, a biosignal may be measured even with low-performance hardware capable of processing signals at a low rate, and power consumption for biosignal measurement may be reduced.

According to an embodiment of the inventive concept, a biosignal may be accurately measured using a pulse signal transmitted through a straight path that passes through a subject with avoidance of signal interference by a multi-path.

The above-disclosed subject matter is to be considered illustrative and not restrictive, and the appended claims are intended to cover all such modifications, enhancements, and other embodiments, which fall within the true spirit and scope of the inventive concept. Thus, to the maximum extent allowed by law, the scope of the inventive concept is to be determined by the broadest permissible interpretation of the following claims and their equivalents, and shall not be restricted or limited by the foregoing detailed description.

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

In the description below, it will be understood that when an element such as a layer, region, substrate, plate, or member is referred to as being “on” another element, it can be directly on the other element or intervening elements may be present. In contrast, the term “directly” means that there are no intervening elements.

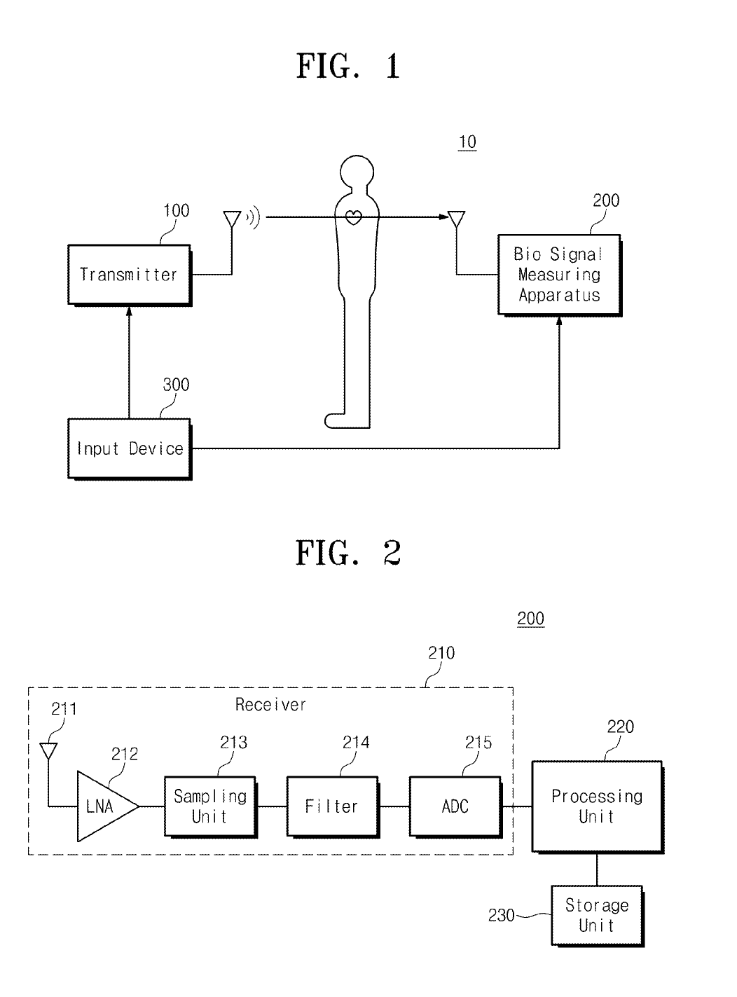

FIG. 1 is a block diagram schematically illustrating a user monitoring system 10 according to an exemplary embodiment of the inventive concept.

As illustrated in FIG. 1, a user monitoring system 10 may contain a transmitter 100 and a bio signal measuring apparatus 200.

The transmitter 100 may generate a pulse signal and may transmit the pulse signal to a user. The bio signal measuring apparatus 200 may be disposed to face the transmitter 100 with the user interposed therebetween.

According to an exemplary embodiment of the inventive concept, the bio signal measuring apparatus 200 may measure a bio signal of the user using a pulse signal that is emitted from the transmitter 100 and penetrates the user.

According to an exemplary embodiment of the inventive concept, the bio signal measuring apparatus 200 may measure a heartbeat using the bio signal of the user, but the measured bio signal may not be limited thereto. As will be described later, as the pulse signal penetrates the user, signal characteristics including a bandwidth may vary. In this case, the bio signal measuring apparatus 200 may measure a condition or movement of an internal organ of the user as well as a heart.

The user monitoring system 10 may measure a bio signal of the user and may monitor a condition of the user based on the measured bio signal.

In exemplary embodiments, the user monitoring system 10 may be installed at a vehicle to monitor conditions of a passenger as well as a driver, but an application field of the user monitoring system 10 may not be limited thereto.

For example, the user monitoring system 10 may be installed at a place (e.g., a cinema or theater) where movement of a user is less, thereby monitoring a condition of the audience.

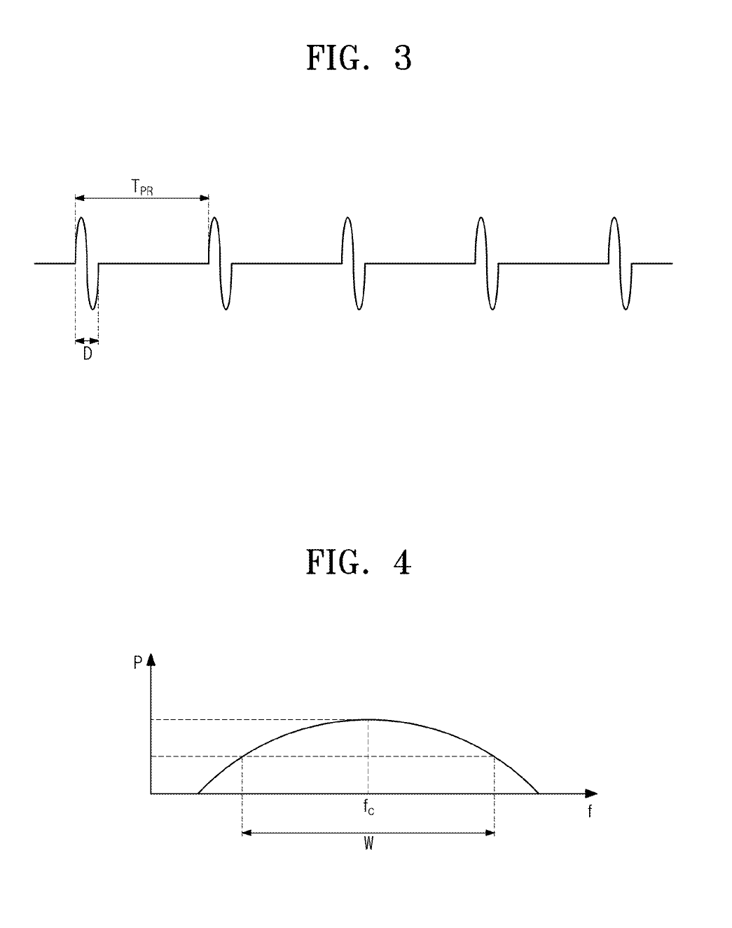

FIG. 2 is a block diagram schematically illustrating a bio signal measuring apparatus 200 according to an exemplary embodiment of the inventive concept.

As illustrated in FIG. 2, a bio signal measuring apparatus 200 may contain a receiver 210, a processing unit 220, and a storage unit 230.

The receiver 210 may receive a pulse signal that is emitted from a transmitter 100 and penetrates a person to be measured. The processing unit 220 may process the received pulse signal to analyze a bandwidth of the pulse signal and may measure a bio signal of the to-be-measured person based on a result of analyzing the bandwidth. The storage unit 230 may store data used to measure the bio signal.

In exemplary embodiments, the receiver 210 may contain an antenna 211, an amplifier 212, a sampling unit 213, and an analog-to-digital converter 215. The antenna 211 may receive a pulse signal that penetrates a user from which a bio signal is measured, that is, a person to be measured.

The amplifier 212 may amplify a received signal, and may be formed of, for example, a low noise amplifier. The sampling unit 213 may sample the amplified signal. The analog-to-digital converter 215 may convert the sampled signal into a digital signal. in exemplary embodiments, the a filter 214 may be further provided between the sampling unit 213 and the analog-to-digital converter 215 to remove unnecessary noise included in a signal at the sampling operation.

The processing unit 220 may process the digital signal to analyze a bandwidth of the pulse signal and may measure a bio signal of a to-be-measured person based on a result of analyzing the bio signal. The processing unit 220 may be a processor that calls and executes, the storage unit 230, a program used to measure a bio signal, and may include, for example, a central processing unit (CPU).

The storage unit 230 may be a storage device that stores a variety of data used to measure the bio signal and may include, for example, a register, a RAM, a ROM, a hard disk drive (HDD), a solid state drive (SSD), and the like.

FIG. 3 is a diagram schematically illustrating a waveform of a time-domain pulse signal used to measure a bio signal of a to-be-measured person, according to an exemplary embodiment of the inventive concept.

Referring to FIG. 3, a pulse signal may be a signal of which the pulse is iterated every pulse iteration period. In exemplary embodiments, the pulse may be an impulse having duration (D) corresponding to a nanosecond unit, and the pulse signal may be a ultra wide band (UWB) in which an impulse is repeated every pulse repetition period T_{PR}.

A pulse signal that is transmitted from a transmitter 100 and penetrates a to-be-measured person may be received by an antenna 211, and the received pulse signal may be amplified by an amplifier 212. The amplified pulse signal may be sampled by a sampling unit 213, and the sampled signal may be converted into a digital signal by an analog-to-digital converter 215.

A processing unit 220 may process the digital signal according to a predetermined process to analyze a bandwidth of the received pulse signal and may measure a bio signal of the to-be-measured person, for example, a heartbeat according to a result of analyzing the bandwidth.

FIG. 4 is a diagram schematically illustrating power spectrum of a pulse signal used to measure a bio signal of a to-be-measured person, according to an exemplary embodiment of the inventive concept.

As described above, since a pulse signal used to measure a bio signal of a to-be-measured person is a signal in which an impulse having very short duration corresponding to a nanosecond unit is repeated, in a frequency domain, low power spectrum density may be distributed over a very wide bandwidth ranging to several gigahertzes as illustrated in FIG. 4.

According to an exemplary embodiment of the inventive concept, a bio signal such as a heartbeat may be measured by transmitting an ultra-wide band signal to a to-be-measured person and analyzing a bandwidth of a signal penetrating the to-be-measured person. Below, a process that a processing unit performs to measure a heartbeat of a bio signal will be more fully described with reference to accompanying drawings.

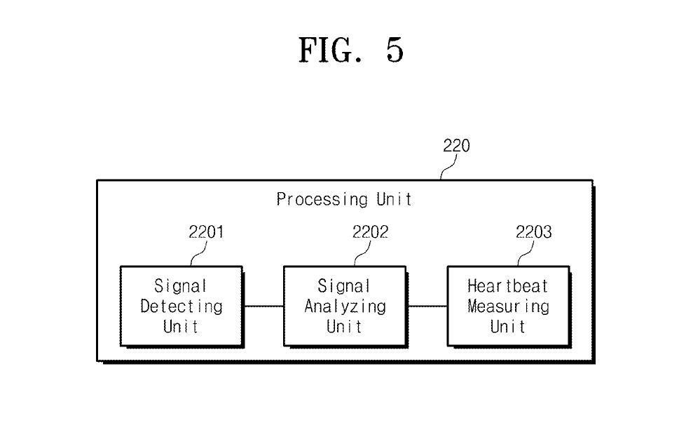

FIG. 5 is a block diagram schematically illustrating a processing unit 220 according to an exemplary embodiment of the inventive concept.

As illustrated in FIG. 5, a processing unit 220 may contain a signal detecting unit 2201, a signal analyzing unit 2202, and a heartbeat measuring unit 2203.

The signal detecting unit 2201 may detect a signal, penetrating a heart of a to-be-measured person, from among a received pulse signal.

Even though a transmitter 100 transmits a pulse signal toward a heart of the to-be-measured person, a portion of the transmitted signal may penetrates an organ different from a heart, for example, a lung and reaches a bio signal measuring apparatus 200.

In this case, the signal detecting unit 2201 may merely detect a signal, penetrating a heart, from among the received pulse signal and may remove a signal penetrating a lung.

In exemplary embodiments, the signal detecting unit 2201 may detect a signal penetrating a heart using the strength of signal.

Since the heart is filled with liquid such as blood and the lung is filled with gas such as air, attenuation of a signal penetrating the heart may be different from that of a signal penetrating the lung.

Since attenuation of a signal penetrating gas is greater than that of a signal penetrating liquid, according to an exemplary embodiment of the inventive concept, the signal detecting unit 2201 may classify a received signal into two groups according to the strength of signal, and may determine a signal belonging to a group of which the strength is small, as a signal penetrating the heart.

In other exemplary embodiments, the signal detecting unit 2201 may detect a signal penetrating the heart using a time when a signal is received.

In detail, the signal detecting unit 2201 may determine a signal, first received, from among signals transmitted at the same time from a transmitter 100, as a signal penetrating a heart.

A signal that the bio signal measuring apparatus 200 first receives may correspond to a signal of which the transmission distance is shortest, and the signal may correspond to a signal that is transmitted in a straight line between a transmitter 100 and the bio signal measuring apparatus 200.

Accordingly, as illustrated in FIG. 1, in the case where the transmitter 100 and the bio signal measuring apparatus 200 are placed on a straight line passing through a heart of a person to be measured, the first received signal may be a signal passing through a heart.

The signal analyzing unit 2203 may analyze a bandwidth of a signal that the signal detecting unit 2201 detects as a signal penetrating a heart. The signal analyzing unit 2203 may measure a heartbeat of the to-be-measured person based on an analysis result of the signal analyzing unit 2202.

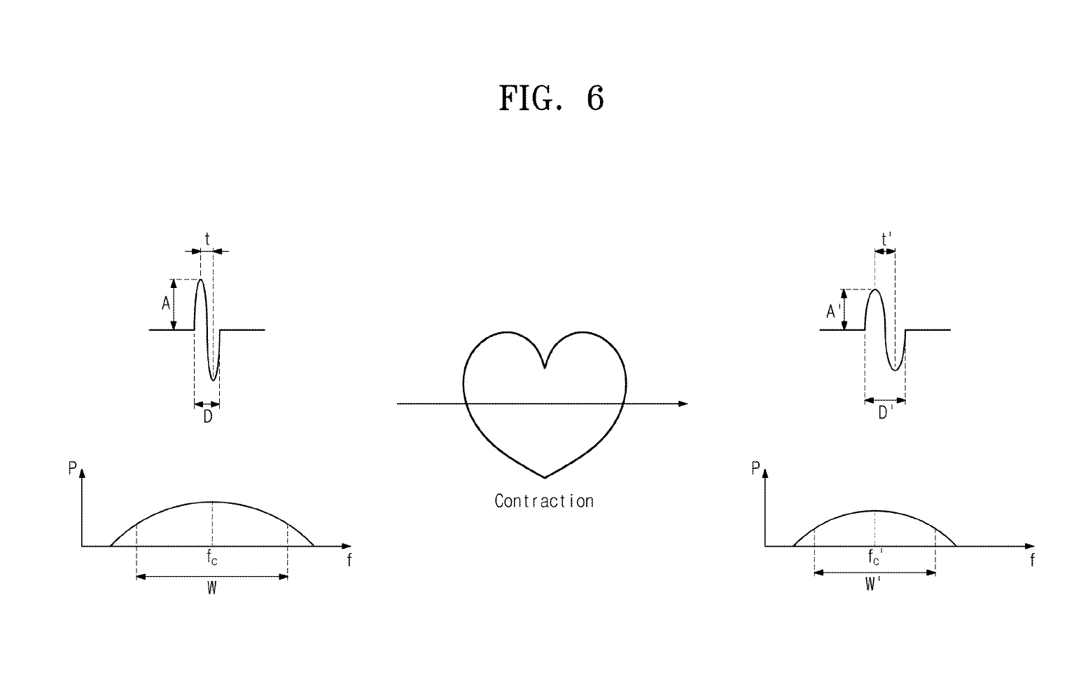

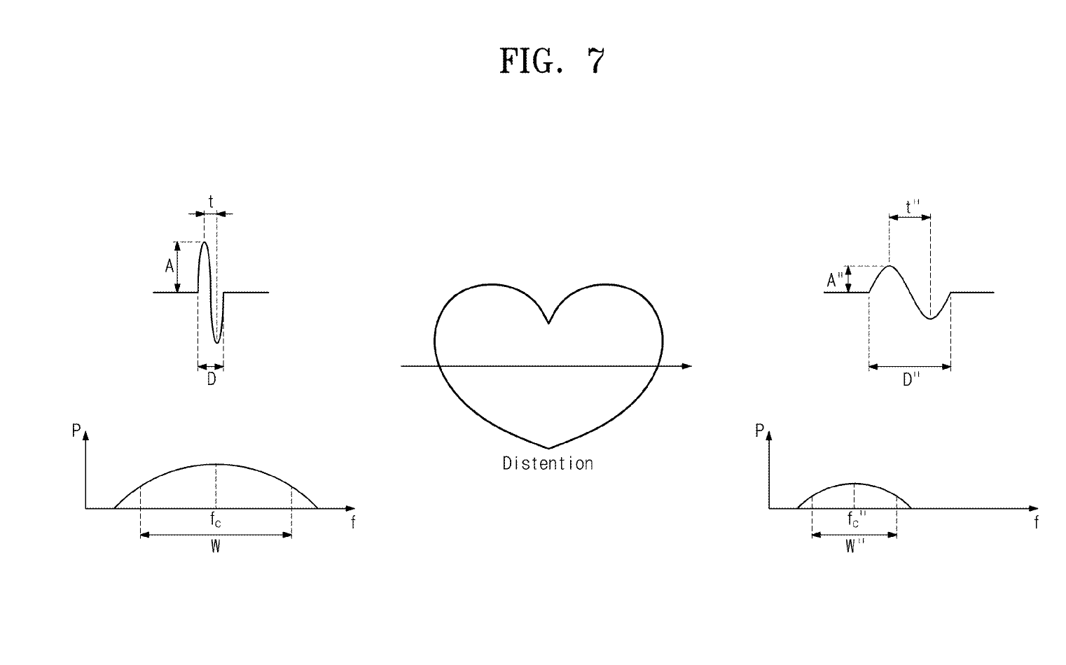

FIGS. 6 and 7 are diagrams schematically illustrating time-domain waveforms and frequency-domain power spectrums after a pulse signal passes through a contracted heart and a dilated heart, according to an exemplary embodiment of the inventive concept.

According to an exemplary embodiment of the inventive concept, in the case where a pulse signal penetrates a heart, a center frequency fc and amplitude A as well as a bandwidth W may decrease, and the decrement may vary according to the size of heart.

For example, as illustrated in FIGS. 6 and 7, it may be assumed that a pulse signal transmitted toward a heart of a to-be-measured person has amplitude A in a time domain and a center frequency fc and a bandwidth W in a frequency domain. Furthermore, it may be assumed that when the size of the heart is reduced due to contraction, the pulse signal transmitted toward the heart has amplitude A′ in a time domain and a center frequency fc′ and a bandwidth W′ in a frequency domain. Furthermore, it may be assumed that when the size of the heart is reduced due to distention, the pulse signal transmitted toward the heart has amplitude A″ in a time domain and a center frequency fc″ and a bandwidth W″ in a frequency domain. According to such assumptions, an amplitude relation between signals may be A>A′>A″, a center frequency relation between the signals may be fc>fc′>fc″, and a bandwidth relation between the signals may be W>W′>W″.

That is, according to an exemplary embodiment of the inventive concept, as the size of heart becomes larger, a center frequency fc of a pulse signal penetrating a heart may become lower, its bandwidth W may become narrower, and its amplitude A may become smaller.

According to an exemplary embodiment of the inventive concept, a processing unit 220 may measure a heartbeat by monitoring a bandwidth W, a center frequency fc, and amplitude A of a received pulse signal to detect contraction and distention of the heart.

In other exemplary embodiments, the processing unit 220 may measure a heartbeat by monitoring variations in a bandwidth W, a center frequency fc, and amplitude A of a received pulse signal to detect contraction and distention of the heart.

Below, an operation in which the processing unit 220 measures a heartbeat using a pulse signal will be more fully described with reference to accompanying drawings.

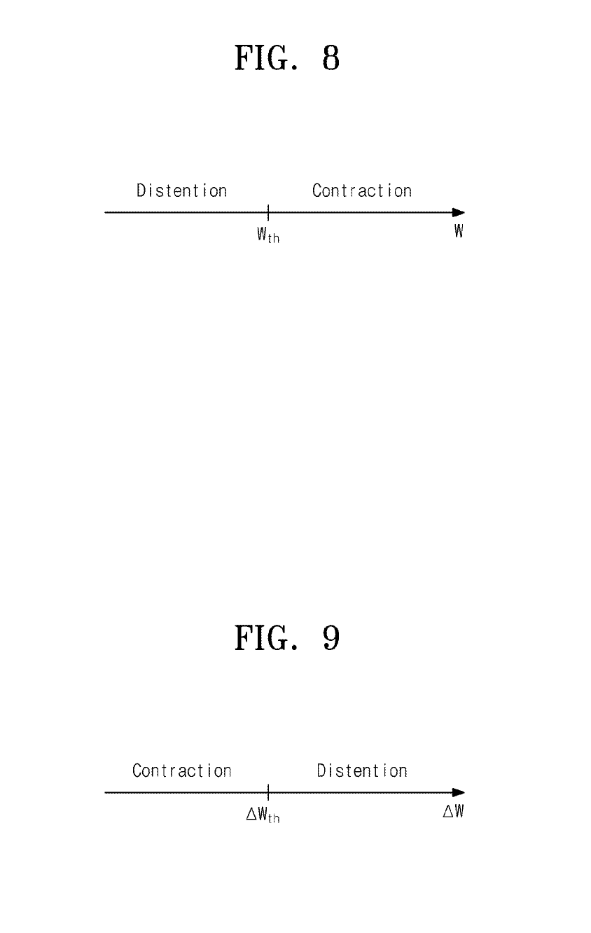

FIG. 8 is a diagram for describing a method for measuring a heartbeat using a bandwidth W of a pulse signal, according to an exemplary embodiment of the inventive concept.

According to an exemplary embodiment of the inventive concept, after calculating a bandwidth W of a pulse signal, a processing unit 220 may compare the bandwidth W with a predetermined threshold value to determine whether a heart is contracted or distended.

In detail, referring to FIG. 8, the processing unit 220 may calculate a bandwidth W of the pulse signal. When the bandwidth W is greater than a predetermined bandwidth threshold value Wₜₕ, the processing unit 220 may determine a period of a pulse signal having the bandwidth, as a contraction period of a heart. When the bandwidth W is smaller than the predetermined bandwidth threshold value Wₜₕ, the processing unit 220 may determine a period of a pulse signal having the bandwidth, as a distention period of a heart.

In other words, the processing unit 220 may determine, as a contraction period of a heart, a period where a bandwidth W of a received pulse signal is greater than a predetermined level and may determine, as a distention period of a heart, a period where the bandwidth W of the received pulse signal is smaller than the predetermined level.

FIG. 9 is a diagram for describing a method for measuring a heartbeat using variations ΔW in bandwidth before and after a pulse signal penetrates a heart, according to an exemplary embodiment of the inventive concept.

In exemplary embodiments, a processing unit 220 may compare bandwidths W before and after a pulse signal passes through a heart, to calculate a variation ΔW in bandwidth. The processing unit 220 may compare the variation ΔW with a predetermined threshold value to determine whether the heart is contracted or distended.

In detail, the processing unit 220 may compare a bandwidth before the pulse signal penetrates a to-be-measured person and a bandwidth after the pulse signal penetrates the to-be-measured person, to calculate the variation ΔW in bandwidth W before and after the pulse signal penetrates the to-be-measured person. When the variation ΔW in bandwidth is smaller than a predetermined bandwidth variation threshold value ΔWₜₕ, a period of the pulse signal having such variation ΔW may be determined as a contraction period of a heart. When the variation ΔW in bandwidth is greater than the bandwidth variation threshold value ΔWₜₕ, a period of the pulse signal having such variation ΔW may be determined as a distention period of a heart.

In other words, the processing unit 220 may determine, as a contraction period of a heart, a period where the variation ΔW in bandwidth before and after the pulse signal penetrates the to-be-measured person is smaller than a predetermined level, and may determine, as a distension period, a period where the variation ΔW in bandwidth is greater than the predetermined level.

A heart filled with blood may have a characteristic of a low pass filter. For this reason, as the size of heart becomes larger, a bandwidth of a pulse signal passing through the heart may become narrower. According to an exemplary embodiment of the inventive concept, a heartbeat of a user may be measured using a bandwidth related characteristic.

In addition, according to another exemplary embodiment of the inventive concept, the processing unit 220 may further analyze at least one of a center frequency fc or amplitude A of a pulse signal and may measure a bio signal of a to-be-measured person, based on an analysis result about the bandwidth W and an analysis result about at least one of the center frequency fc or the amplitude A.

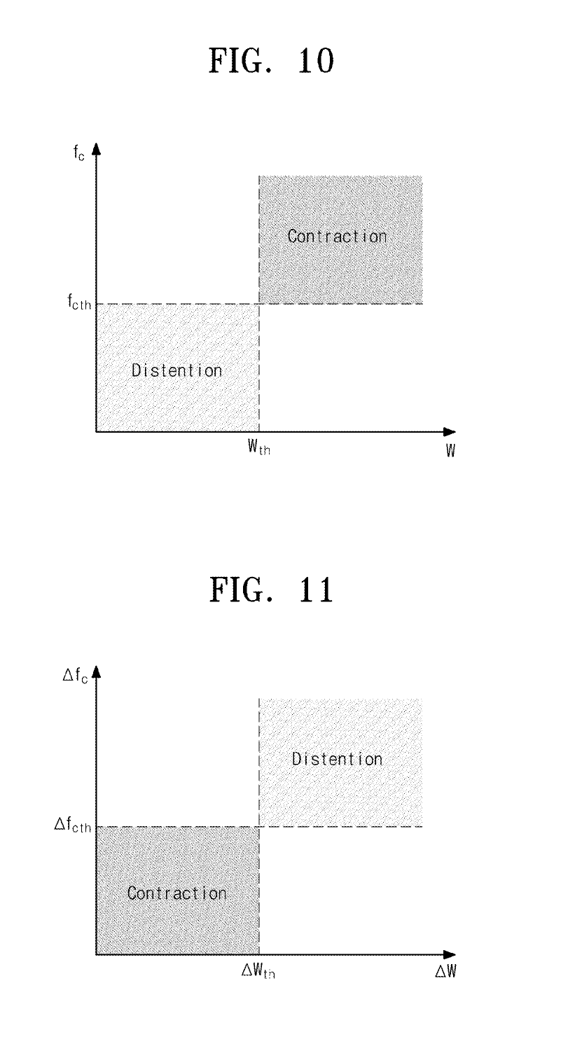

FIG. 10 is a diagram for describing a method for measuring a heartbeat using a bandwidth W and a center frequency fc of a pulse signal, according to an exemplary embodiment of the inventive concept.

In exemplary embodiments, a processing unit 220 may further analyze a center frequency fc of a pulse signal as well as a bandwidth W thereof and may determine whether a heart is contracted or distended, based on the analysis result.

In detail, the processing unit 220 may calculate the center frequency fc of a pulse signal. When the center frequency fc is higher than a predetermined center frequency threshold value f_{cth}, the processing unit 220 may determine, as a distension period of a heart, a period where a pulse signal has the center frequency fc. When the center frequency fc is lower than the center frequency threshold value f_{cth}, the processing unit 220 may determine, as a contraction period of a heart, a period where a pulse signal has the center frequency fc.

In exemplary embodiments, the processing unit 220 may more accurately determine whether a heart is contracted or distended, by measuring a heartbeat using a center frequency fc of a received pulse signal as well as a bandwidth W thereof.

For example, as illustrated in FIG. 10, the processing unit 220 may determine, a contraction period of a heart, a period where a bandwidth W of a pulse signal is greater than a bandwidth threshold value Wₜₕ and a center frequency fc is higher than a center frequency threshold value f_{cth}. In contrast, the processing unit 220 may determine, a distension period of a heart, a period where a bandwidth W of a pulse signal is smaller than the bandwidth threshold value Wₜₕ and a center frequency fc is lower than the center frequency threshold value f_{cth}.

As such, conditions used to determine whether a heart is contracted or distended may increase, thereby reducing an error occurring in measuring a heartbeat and measuring a heartbeat more accurately.

In exemplary embodiments, the processing unit 220 may grade a score based on a difference between a bandwidth W of a received pulse signal and a bandwidth threshold value Wₜₕ and may grade a score based on a difference between a center frequency fc of the received pulse signal and a center frequency threshold value f_{cth}. Next, the processing unit 220 may assign the same or different weights to the score about the bandwidth W and the score about the center frequency fc and may determine whether a heart is contrasted or distended, using a final score obtained by adding the weighted scores. In this case, the reliability of measurement may be enhanced by assigning a greater weight to one, important to measure a heartbeat, from among the bandwidth W and the center frequency fc.

FIG. 11 is a diagram for describing a method for measuring a heartbeat using a bandwidth variation ΔW and a center frequency variation Δfc of a pulse signal, according to another exemplary embodiment of the inventive concept.

In exemplary embodiments, a processing unit 220 may further analyze a center frequency variation Δfc of a pulse signal as well as a bandwidth variation ΔW thereof and may determine whether a heart is contrasted or distended, based on the analysis result.

In detail, the processing unit 220 may compare center frequencies before and after a pulse signal passes through a to-be-measured person, to calculate a center frequency variation Δfc before and after the pulse signal passes through the to-be-measured person. When the variation Δfc in the center frequency is smaller than a predetermined center frequency variation threshold value Δf_{cth}, the processing unit 220 may determine, as a contraction period of a heart, a period where the pulse signal has the variation. When the variation Δfc in the center frequency is greater than the center frequency variation threshold value Δf_{cth}, the processing unit 220 may determine, as a distension period of a heart, a period where the pulse signal has the variation.

In exemplary embodiments, the processing unit 220 may more accurately determine whether a heart is contracted or distended, by measuring a heartbeat using a center frequency variation Δfc of a received pulse signal as well as a bandwidth variation ΔW thereof.

For example, as illustrated in FIG. 11, the processing unit 220 may determine, as a contraction period of a heart, a period where a bandwidth variation ΔW of a pulse signal is smaller than a bandwidth variation threshold value ΔWₜₕ and a center frequency variation Δfc is smaller than a center frequency variation threshold value Δf_{cth}. In contrast, the processing unit 220 may determine, as a distension period of a heart, a period where the bandwidth variation ΔW of the pulse signal is greater than the bandwidth variation threshold value ΔWₜₕ and the center frequency variation Δfc is greater than the center frequency variation threshold value Δf_{cth}.

In exemplary embodiments, the processing unit 220 may grade a score based on a difference between a bandwidth variation ΔW of a received pulse signal and a bandwidth variation threshold value ΔWₜₕ and may grade a score based on a difference between a center frequency variation Δfc of the received pulse signal and a center frequency variation threshold value Δf_{cth}. Next, the processing unit 220 may assign the same or different weights to the score about the bandwidth variation ΔW and the score about the center frequency variation Δfc and may determine whether a heart is contrasted or distended, using a final score obtained by adding the weighted scores. In this case, the reliability of measurement may be enhanced by assigning a greater weight to one, important to measure a heartbeat, from among the bandwidth variation ΔW and the center frequency variation Δfc.

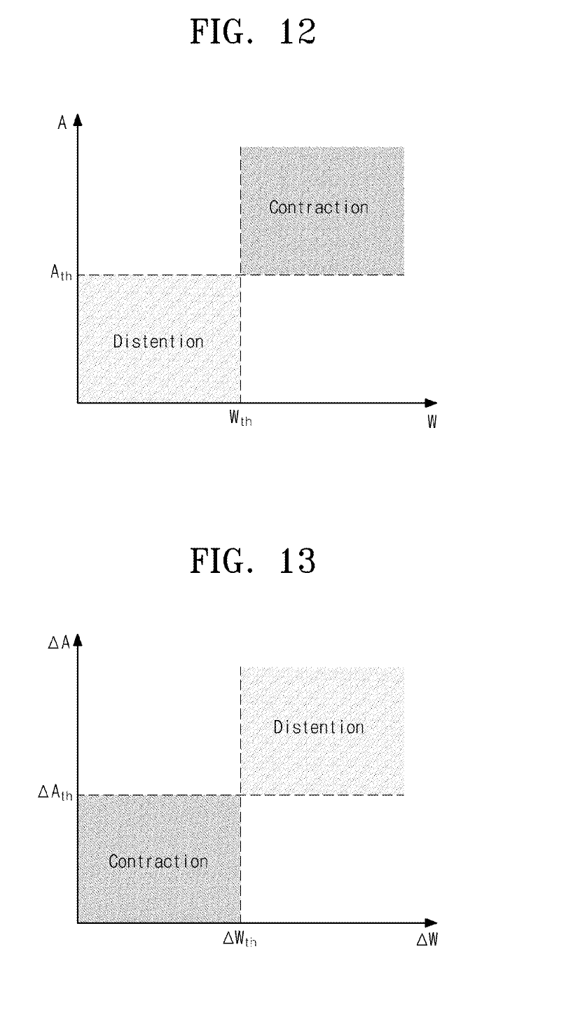

FIG. 12 is a diagram for describing a method for measuring a heartbeat using a bandwidth W and amplitude A of a pulse signal, according to still another exemplary embodiment of the inventive concept.

In exemplary embodiments, a processing unit 220 may further analyze amplitude A of a pulse signal as well as a bandwidth W thereof and may determine whether a heart is contrasted or distended, based on the analysis result.

In detail, the processing unit 220 may calculate amplitude A of the pulse signal. When the amplitude A is greater than a predetermined amplitude threshold value Aₜₕ, the processing unit 220 may determine, as a contraction period of a heart, a period where the pulse signal has the amplitude. When the amplitude A is smaller than the amplitude threshold value Aₜₕ, the processing unit 220 may determine, as a distension period of a heart, a period where the pulse signal has the amplitude.

In exemplary embodiments, the processing unit 220 may more accurately determine whether a heart is contracted or distended, by measuring a heartbeat using amplitude A of a received pulse signal as well as a bandwidth W thereof.

For example, as illustrated in FIG. 12, the processing unit 220 may determine, as a contraction period of a heart, a period where a bandwidth W of a pulse signal is greater than a bandwidth threshold value Wₜₕ and amplitude A is greater than an amplitude threshold value Aₜₕ. In contrast, the processing unit 220 may determine, as a distension period of a heart, a period where the bandwidth W of the pulse signal is smaller than the bandwidth threshold value Wₜₕ and the amplitude A is smaller than the amplitude threshold value Aₜₕ.

In exemplary embodiments, the processing unit 220 may grade a score based on a difference between a bandwidth W of a received pulse signal and a bandwidth threshold value Wₜₕ and may grade a score based on a difference between amplitude A of the received pulse signal and an amplitude threshold value Aₜₕ. Next, the processing unit 220 may assign the same or different weights to the score about the bandwidth W and the score about the amplitude A and may determine whether a heart is contrasted or distended, using a final score obtained by adding the weighted scores. In this case, the reliability of measurement may be enhanced by assigning a greater weight to one, important to measure a heartbeat, from among the bandwidth W and the amplitude A.

FIG. 13 is a diagram for describing a method for measuring a heartbeat using a bandwidth variation ΔW and an amplitude variation ΔA of a pulse signal, according to still another exemplary embodiment of the inventive concept.

In exemplary embodiments, a processing unit 220 may further analyze an amplitude variation ΔA of a pulse signal as well as a bandwidth variation ΔW thereof and may determine whether a heart is contrasted or distended, based on the analysis result.

In detail, the processing unit 220 may compare amplitudes of a pulse signal before and after the pulse signal passes through a heart of a to-be-measured person, to calculate an amplitude variation ΔA. When the amplitude variation ΔA is smaller than a predetermined amplitude variation threshold value ΔAₜₕ, the processing unit 220 may determine, as a contraction period of a heart, a period where the pulse signal has the amplitude variation ΔA. When the amplitude variation ΔA is greater than the amplitude variation threshold value ΔAₜₕ, the processing unit 220 may determine, as a distension period of a heart, a period where the pulse signal has the amplitude variation ΔA.

In exemplary embodiments, the processing unit 220 may more accurately determine whether a heart is contracted or distended, by measuring a heartbeat using an amplitude variation ΔA of a received pulse signal as well as a bandwidth variation ΔW thereof.

For example, as illustrated in FIG. 13, the processing unit 220 may determine, as a contraction period of a heart, a period where a bandwidth variation ΔW of a pulse signal is smaller than a bandwidth variation threshold value ΔWₜₕ and an amplitude variation ΔA is smaller than an amplitude variation threshold value ΔAₜₕ. In contrast, the processing unit 220 may determine, as a distension period of a heart, a period where the bandwidth variation ΔW of the pulse signal is greater than the bandwidth variation threshold value ΔWₜₕ and the amplitude variation ΔA is greater than the amplitude variation threshold value ΔAₜₕ.

In exemplary embodiments, the processing unit 220 may grade a score based on a difference between a bandwidth variation ΔW of a received pulse signal and a bandwidth variation threshold value ΔWₜₕ and may grade a score based on a difference between an amplitude variation ΔA of the received pulse signal and an amplitude variation threshold value ΔAₜₕ. Next, the processing unit 220 may assign the same or different weights to the score about the bandwidth variation ΔW and the score about the amplitude variation ΔA and may determine whether a heart is contrasted or distended, using a final score obtained by adding the weighted scores. In this case, the reliability of measurement may be enhanced by assigning a greater weight to one, important to measure a heartbeat, from among the bandwidth variation ΔW and the amplitude variation ΔA.

Embodiments of the inventive concept may be illustrated as a heartbeat is measured using a result of analyzing a center frequency fc or amplitude A of a pulse signal as well as a result of analyzing a bandwidth W thereof or a result of analyzing a center frequency variation Δfc or an amplitude variation ΔA as well as a result of analyzing a bandwidth variation ΔW. However, the scope and spirit of the inventive concept may not be limited thereto. For example, a heartbeat may be measured using a bandwidth W, a center frequency fc, and amplitude A, using a bandwidth variation ΔW, a center frequency variation Δfc, and an amplitude variation ΔA, or using a combination of two or more of the bandwidth W, the center frequency fc, the amplitude A, the bandwidth variation ΔW, the center frequency variation Δfc, and the amplitude variation ΔA.

According to an exemplary embodiment of the inventive concept, the processing unit 220 may convert a received pulse signal from a time domain to a frequency domain and may measure a center frequency or a bandwidth of a pulse signal in the time domain. At this time, the processing unit 220 may obtain a frequency spectrum of pulse signal using a Fourier transform algorithm. However, the scope and spirit of the inventive concept may not be limited thereto.

According to another exemplary embodiment of the inventive concept, the processing unit may process a received pulse signal in a time domain without converting into a frequency domain to analyze a bandwidth or a center frequency.

In exemplary embodiments, the processing unit 220 may measure duration D of a pulse included in a pulse signal in a time domain and may analyze a bandwidth W of a pulse signal using the duration D.

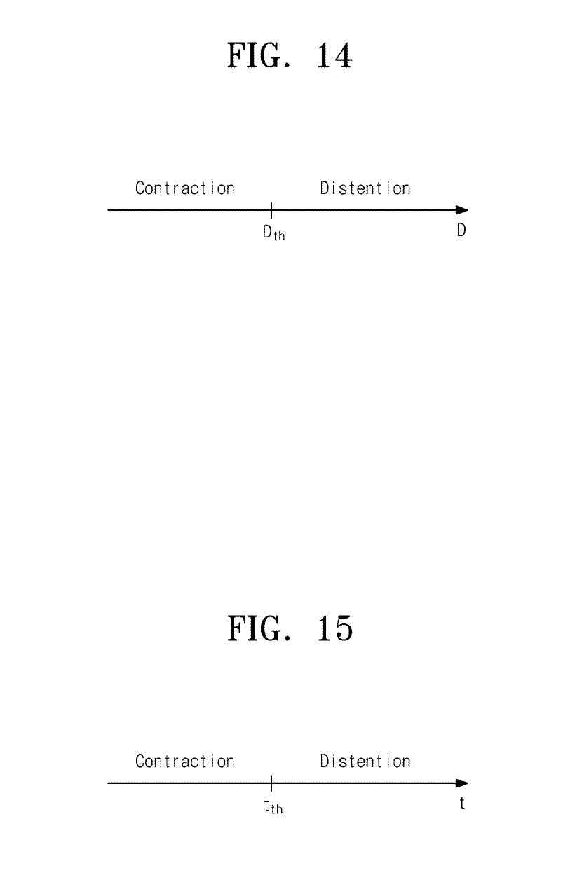

FIG. 14 is a diagram for describing a method for measuring a heartbeat by analyzing a bandwidth W using duration D of a pulse signal, according to still another exemplary embodiment of the inventive concept.

For example, referring to FIG. 14, a processing unit 220 may compare duration D with a duration threshold value Dₜₕ corresponding to the above-described bandwidth threshold value Wₜₕ. When the duration D is shorter than the bandwidth threshold value Wₜₕ, a period where a pulse signal has the duration may be determined as a contraction period of a heart. When the duration D is longer than the bandwidth threshold value Wₜₕ, a period where a pulse signal has the duration may be determined as a distension period of a heart.

Furthermore, the processing unit 220 may measure duration D of a pulse included in a pulse signal in a time domain and may compare durations before and after the pulse signal passes through a to-be-measured person, to calculate a duration variation ΔD before and after the pulse signal passes through a to-be-measured person. Next, the processing unit 220 may analyze a bandwidth variation ΔW using the duration variation ΔD.

For example, the processing unit 220 may compare the duration variation ΔD with a duration variation threshold value ΔDth corresponding to the bandwidth variation threshold value ΔWₜₕ. When the duration variation ΔD is smaller than the duration variation threshold value ΔDth, the processing unit 220 may determine, as a contraction period of a heart, a period where a pulse signal has the duration variation ΔD. When the duration variation ΔD is greater than the duration variation threshold value ΔDth, the processing unit 220 may determine, as a distension period of a heart, a period where a pulse signal has the duration variation ΔD.

Furthermore, the processing unit 220 may analyze not only a bandwidth W based on duration D of a pulse signal, but it may analyze a center frequency fc based on a time difference t (in FIGS. 6 and 7) between a pulse portion having positive amplitude and pulse portion having negative amplitude.

FIG. 15 is a diagram for describing a method for measuring a heartbeat by analyzing a center frequency fc using a time difference t between a pulse portion having positive amplitude and a pulse portion having negative amplitude, according to a further exemplary embodiment of the inventive concept.

For example, referring to FIG. 15, a processing unit 220 may measure a time difference t of a pulse signal in a time domain and may compare the measured time difference t with a time difference threshold value tₜₕ corresponding to the above-described center frequency threshold value f_{cth}. When the time difference t is smaller than the time difference threshold value tₜₕ, the processing unit 220 may determine, as a contraction period of a heart, a period where the pulse signal has the time difference t. When the time difference t is greater than the time difference threshold value tₜₕ, the processing unit 220 may determine, as a distension period of a heart, a period where the pulse signal has the time difference t.

Also, the processing unit 220 may measure the time difference t in the time domain and may calculate a time difference variation Δt by comparing time differences before and after the pulse signal penetrates a person to be measured. The processing unit 220 may analyze a variation Δfc in a center frequency fc using the variation Δt in the time difference.

For example, the processing unit 220 may compare the time difference variation Δt with a time difference variation threshold value Δtₜₕ corresponding to the above-described center frequency variation threshold value Δf_{cth}. When the time difference variation Δt is smaller than the time difference variation threshold value Δtₜₕ, the processing unit 220 may determine, as a contraction period of a heart, a period where the pulse signal has the time difference variation Δt. When the time difference variation Δt is greater than the time difference variation threshold value Δtₜₕ, the processing unit 220 may determine, as a distension period of a heart, a period where the pulse signal has the time difference variation Δt.

As such, a heartbeat may be measured by directly measuring duration D or a time difference t in a time domain, not converting a received pulse signal into a frequency-domain signal to measure a bandwidth W or a center frequency fc, thereby processing a signal more simply and making it easy to implement a system.

A bio signal measuring apparatus and a user monitoring system according to an exemplary embodiment of the inventive concept may perform measurement of a bio signal of a user including a heartbeat and monitoring of a user condition, using a bandwidth of a pulse signal penetrating a body of the user. Accordingly, a bio signal may be accurately measured without influence of movement of the user.

Furthermore, according to an embodiment of the inventive concept, power consumption may be reduced by measuring a bio signal with low-performance hardware for processing a signal in a low speed in measuring a bio signal of a to-be-measured person using a high-speed pulse signal.

To this end, returning to FIG. 2, the sampling unit 213 may sample a portion of a pulse included in the pulse signal such that portions respectively sampled from a plurality of pulses are different.

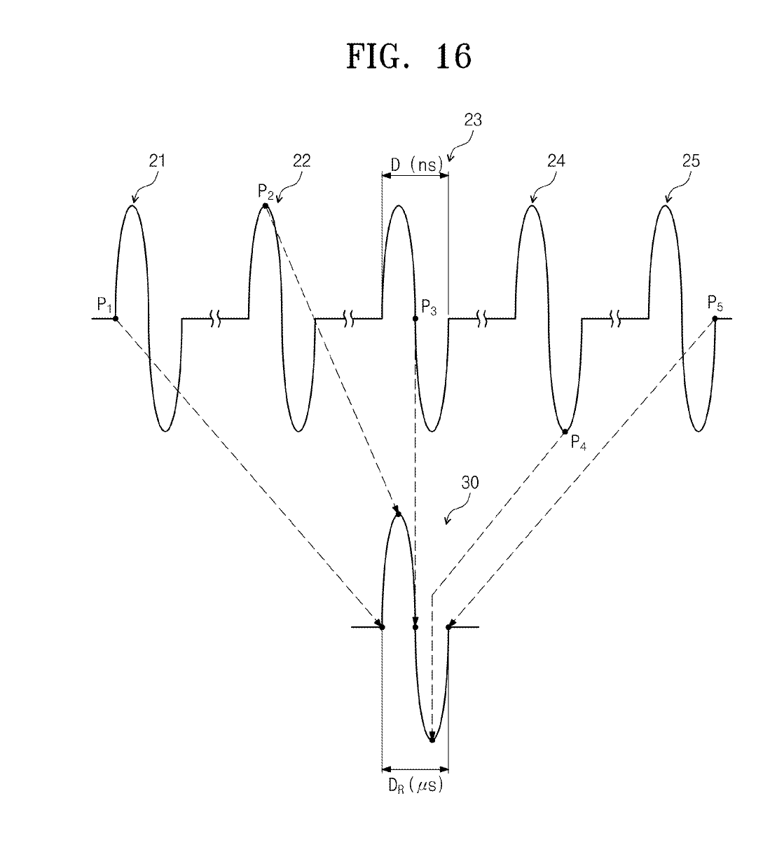

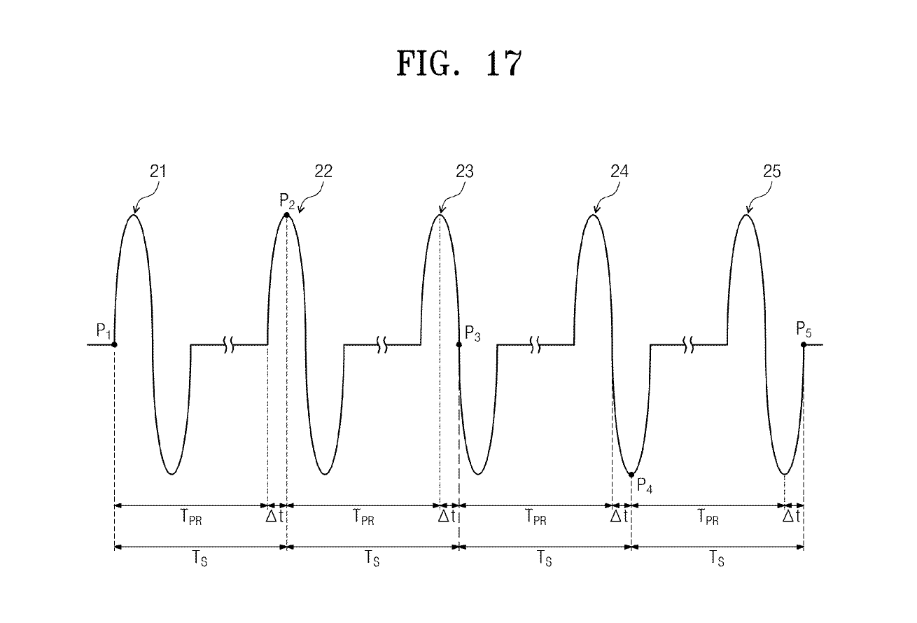

FIGS. 16 and 17 are diagrams for describing a method for a sampling unit 213 sampling a pulse signal, according to an exemplary embodiment of the inventive concept.

A sampling unit 213 may sample a pulse included in a pulse signal partially, not overall.

For example, referring to FIG. 16, the sampling unit 213 may sample portions P1 to P5 of five pulses 21 to 25 included in a pulse signal. At this time, points where the pulses 21 to 25 are respectively sampled may be different from each other.

In detail, a point where a first pulse 21 of the pulse signal is sampled may be a start point P1 of the pulse. A second pulse 22 of the pulse signal is sampled may be a point P2 corresponding to a quarter of duration D of the pulse. A third pulse 23 of the pulse signal is sampled may be a point P3 corresponding to half the duration D of the pulse. A fourth pulse 24 of the pulse signal is sampled may be a point P4 corresponding to three-quarters of duration D of the pulse. A fifth pulse 25 of the pulse signal is sampled may be an end point P5 of the pulse.

As such, the sampling unit 213 may sample a portion of each pulse, not the entirety of each pulse included in a pulse signal. In particular, the sampling unit 213 may sample different portions of the pulses.

Accordingly, a reconstruction pulse 30 reconstructed by sampling may have longer duration D_{R} in comparison with pulses 21 to 25 before sampling so as to be converted into a low-speed signal.

For example, as illustrated in FIG. 16, if pulses 21 to 25 used to measure a bio signal of a to-be-measured person is a high-speed impulse having duration D corresponding to a nanosecond unit, the reconstruction pulse 30 reconstructed by sampling may be converted into a low-speed signal having duration D_{R} corresponding to a microsecond unit.

According to an exemplary embodiment of the inventive concept, sampling of the pulses may be performed every predetermined time, which is obtained by adding a predetermined sampling interval to a pulse repetition period of a pulse signal.

For example, referring to FIG. 17, sampling of each pulse may be performed every T_{S}, a time obtained by adding a sampling interval Δt to a pulse repetition period T_{PR} of a pulse signal.

Accordingly, the sampling unit 213 may sample different portions P1 to P5 of pulses 21 to 25, that is, may perform sampling at different times of duration D.

In exemplary embodiments, the sampling interval Δt may be predetermined and may then be set to a bio signal measuring apparatus 200. Furthermore, the pulse repetition period T_{PR} may be predetermined and may be then set to a transmitter 100 and the bio signal measuring apparatus 200. In exemplary embodiments, the bio signal measuring apparatus 200 may be provided with information about the pulse repetition period T_{PR} from the transmitter 100.

In FIGS. 16 and 17, one reconstruction pulse 30 may be obtained by sampling five pulses 21 to 25 under a condition where the sampling interval Δt is set to a quarter of duration D. However, the scope and spirit of the inventive concept may not be limited thereto. For example, duration D_{R} of a reconstruction pulse may be adjusted by increasing or decreasing the sampling interval Δt.

For example, in the case where the sampling interval Δt is set to three-quarters of the duration D, the duration D_{R} of the reconstruction pulse may become shorter than that of the reconstruction pulse 30 illustrated in FIG. 16. In the case where the sampling interval Δt is set to one-eighth of the duration D, the duration D_{R} of the reconstruction pulse may become longer than that of the reconstruction pulse 30 illustrated in FIG. 16.

Furthermore, in FIGS. 16 and 17, the sampling unit 213 may be illustrated as sampling a pulse at a time of duration of the pulse. However, the number of sampling per pulse may be changed to two or more, not limited to one.

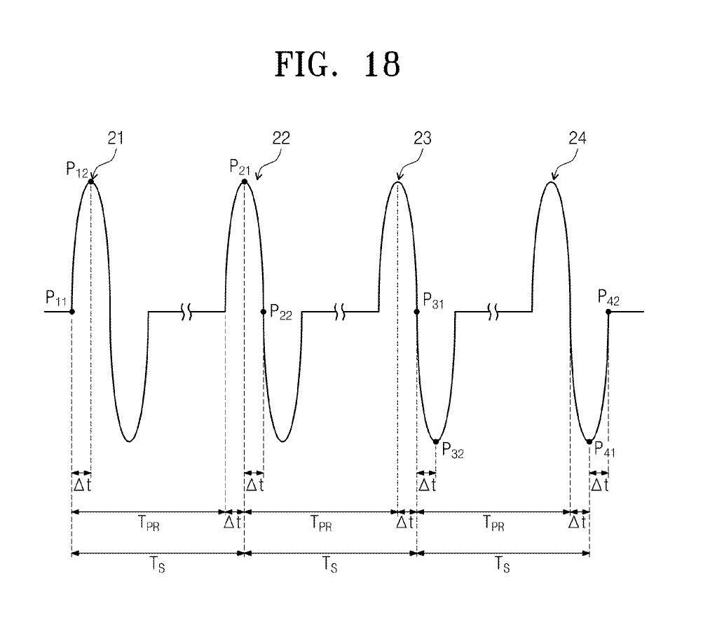

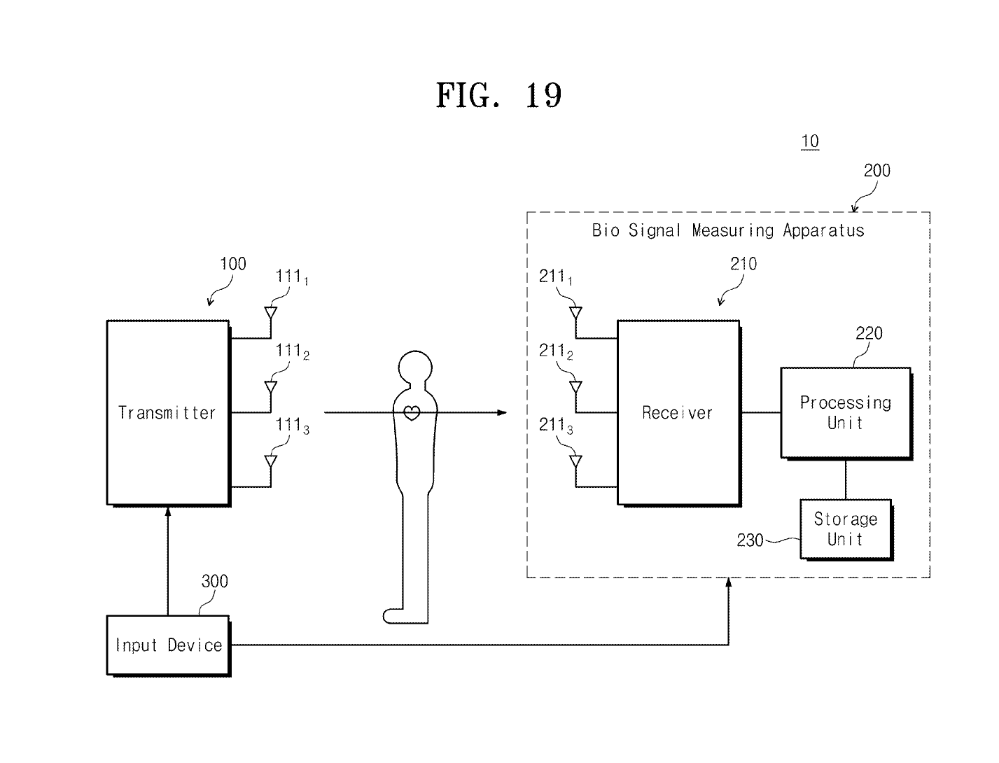

FIG. 18 is a diagram for describing a method in which a sampling unit 213 samples a pulse signal, according to another exemplary embodiment of the inventive concept.

According to another exemplary embodiment of the inventive concept, a sampling unit 213 may sample a pulse at a plurality of times of pulse duration D.

For example, as illustrated in FIG. 18, the sampling unit 213 may sample a pulse two times, and each pulse may be sampled every T_{S}, a time obtained by adding a sampling interval Δt to a pulse repetition period T_{PR}.

Accordingly, a first pulse 21 of a pulse signal may be sampled at a start point P11 of a pulse and a point P12 corresponding to a quarter of duration D. A second pulse 22 may be sampled at a point P21 corresponding to a quarter of the duration D and a point P22 corresponding to half the duration D. A third pulse 23 may be sampled at a point P31 corresponding to half the duration D and a point P32 corresponding to third-quarters of the duration D. A fourth pulse 24 may be sampled at a point P41 corresponding to third-quarters of the duration D and an end point P42 of the pulse.

In exemplary embodiments, intervals between sampling points in the pulse duration D may be the same as the sampling interval Δt.

For example, referring to FIG. 18, an interval between sampling points P11 and P12 in the duration D of the first pulse 21 may correspond to a quarter of the duration D, that is, may be the same as the sampling interval Δt. Likewise, an interval between sampling points in the duration D of each of the second to fourth pulses 22 to 24 may correspond to a quarter of the duration D, that is, may be the same as the sampling interval Δt.

As such, in the case where the sampling unit 213 samples a pulse at a plurality of times of pulse duration D, a processing unit 220 may determine an average of sampling values, sampled at different points in the pulse duration D, from among sampling values obtained by sampling a plurality of pulses, as a sampling value of a corresponding point.

For example, referring to FIG. 18, the processing unit 220 may average sampling values, sampled at different points in the pulse duration D, for example, P12 and P21 corresponding to a quarter of the pulse duration D, from among eight sampling values obtained by sampling the first to fourth pulses 21 to 24 and may determine the average value as a sampling value of the point.

Likewise, the processing unit 220 may average sampling values sampled at different points P22 and P31 corresponding to half the pulse duration D and may determine the average value as a sampling value of the point. Sampling values of other points may be determined according to the above-described method.

According to the above-described embodiment, a signal to noise ratio (SNR) of a reconstruction pulse obtained from sampling may increase, thus improving measurement reliability about a bio signal.

In FIG. 18, an embodiment of the inventive concept is exemplified as the sampling unit 213 samples a pulse at two times of the pulse duration D. However, the scope and spirit of the inventive concept may not be limited thereto. For example, the number of sampling per pulse may be set to three or more, not limited to two. In this case, a signal to noise ratio (SNR) of a reconstruction pulse may be further improved.

The inventive concept, the sampling unit 213 may be implemented with a mixer including a switch that is closed every sampling period T_{S} (=T_{PR}+Δt) or with a sampler further including a capacitor. However, the scope and spirit of the inventive concept may not be limited thereto.

Furthermore, as will be described later, measurement accuracy may be improved by excluding interference due to reflected signals transmitted through a multi-path and using a pulse signal transmitted through a direct path between a transmitter and a receiver, in measuring a bio signal.

FIG. 19 is a block diagram schematically illustrating a user monitoring system 10 according to another exemplary embodiment of the inventive concept.

As illustrated in FIG. 19, a user monitoring system 10 may contain a transmitter 100 and a bio signal measuring apparatus 200. The transmitter 100 may contain at least one antenna 1111, 1112, and 1113, and a receiver 210 of the bio signal measuring apparatus 200 may include at least one antenna 2111, 2112, and 2113.

Below, the inventive concept will be described as each of the transmitter 100 and the receiver 210 has a plurality of antennas. In some embodiments, however, one of the transmitter 100 and the receiver 210 may include one antenna. Furthermore, each of the transmitter 100 and the receiver 210 may have an antenna.

The transmitter 100 may transmit pulse signals through a plurality of antennas 1111, 1112, and 1113 at different times. The receiver 210 may receive and reconstruct a pulse signal penetrating a to-be-measured person through a plurality of antennas 2111, 2112, and 2113.

Furthermore, the bio signal measuring apparatus 200 may include a processing unit 220, and the processing unit 220 may process the reconstructed pulse signal to analyze at least one of a bandwidth, a center frequency, or amplitude of the pulse signal and may measure a bio signal of the to-be-measured person based on the analysis result.

A method in which the processing unit 220 measures a bio signal of a to-be-measured person, for example, a heartbeat using the bandwidth, the center frequency, or the amplitude of the pulse signal may be the same as that described with reference to FIGS. 5 to 15.

Below, the transmitter 100 and the receiver 210 illustrated in FIG. 19 will be described first of all. In the case where the user monitoring system 10 is installed at a vehicle to monitor passengers, arrangements and applications about an antenna array included in the transmitter 100 and an antenna array included in the receiver 210 will be described.

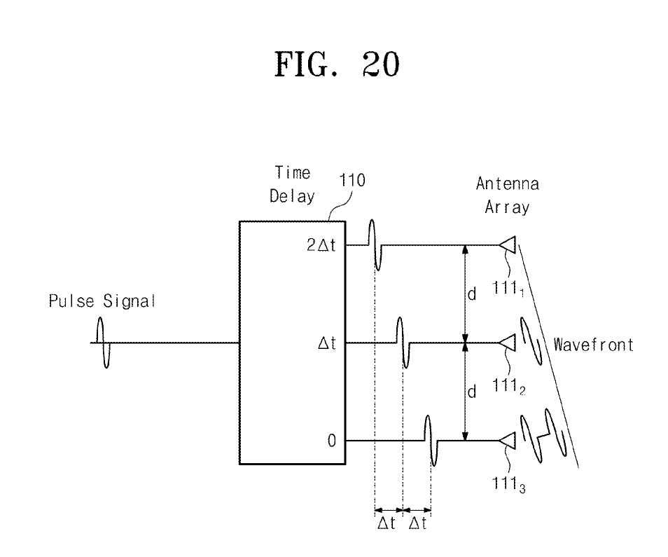

FIG. 20 is a diagram for describing a method in which a transmitter 100 transmits pulse signals, according to another exemplary embodiment of the inventive concept.

As illustrated in FIG. 20, a transmitter 100 may transmit pulse signals through a plurality of antennas 1111, 1112, and 1113 at different times.

The transmitter 100 may contain a delay unit 110 to transmit pulse signals through the antennas 1111, 1112, and 1113 at different times.

In exemplary embodiments, the delay unit 110 may provide the v with pulse signals obtained by delaying a pulse signal by different delay times.

For example, referring to FIG. 20, the delay unit 110 may apply different time delays, for example, 2Δt, Δt, and 0 to a pulse signal provided through RF feed and may provide the delayed pulse signals to the antennas 1111, 1112, and 1113, respectively.

In this case, a signal propagation direction, that is, a wavefront may be determined according to a distance d between antennas and a time difference Δt between pulse signals transmitted through antennas.

According to an exemplary embodiment of the inventive concept, a wavefront of a pulse signal transmitted from the transmitter 100 may be set to face the receiver 210. That is, a user monitoring system 10 may previously determine positions of the transmitter 100 and the receiver 210, a distance d between antennas, and a time difference Δt between pulse signals to allow a measuring part of a to-be-measured person, for example, a heart to be located on a straight line between the transmitter 100 and the receiver 210.

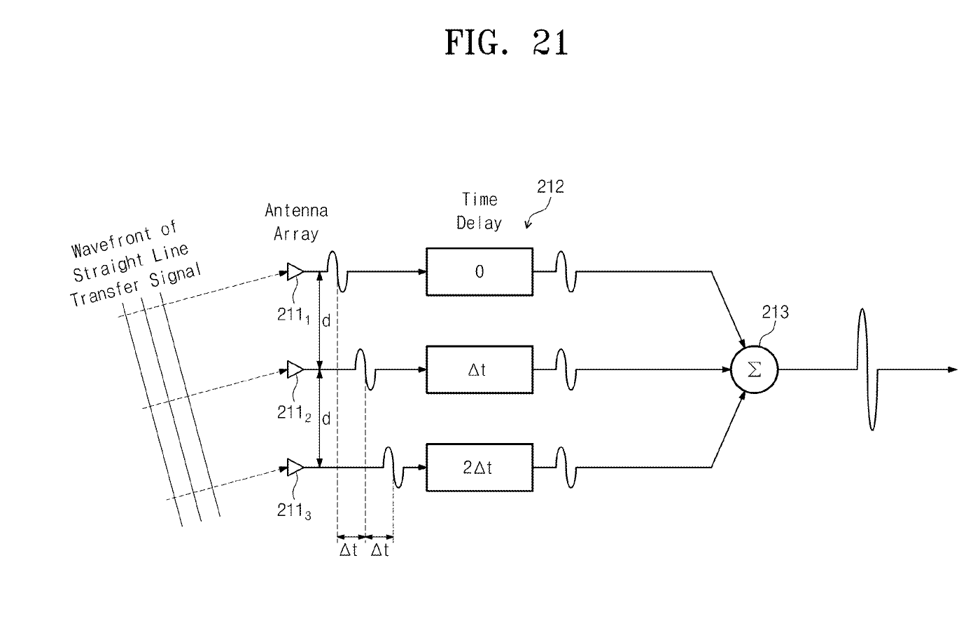

FIG. 21 is a diagram for describing a method in which a receiver 210 receives a pulse signal, according to another exemplary embodiment of the inventive concept.

As illustrated in FIG. 21, pulse signals transmitted through straight line paths may be received through a plurality of antennas 2111, 2112, and 2113. According to an exemplary embodiment of the inventive concept, a receiver 210 may contain a delay unit 212 and an adder 213, to obtain a pulse signal, to be used to measure a bio signal, from among the pulse signals received.

The delay unit 212 may apply different delays to the pulse signals which the antennas 2111, 2112, and 2113 of the receiver 210 receive. The adder 213 may add a plurality of pulse signals that the delay unit 212 outputs.

Referring to FIG. 21, a pulse signal received through each antenna may be delayed by a delay applied to a corresponding pulse signal of a transmitter 100, and the delay unit 212 may apply delays used at the transmitter 100, that is, 0, Δt, and 2Δt to the pulse signals, respectively. Phases of pulse signals transmitted through straight line paths between the transmitter 100 and the receiver 210 may be adjusted so as to have the same values.

Afterwards, the adder 213 may reconstruct a pulse signal by adding phase-adjusted pulse signals from the delay unit 212. In exemplary embodiments, constructive interference may occur due to phase adjustment and adding about pulse signals transmitted through straight line paths in the receiver 210.

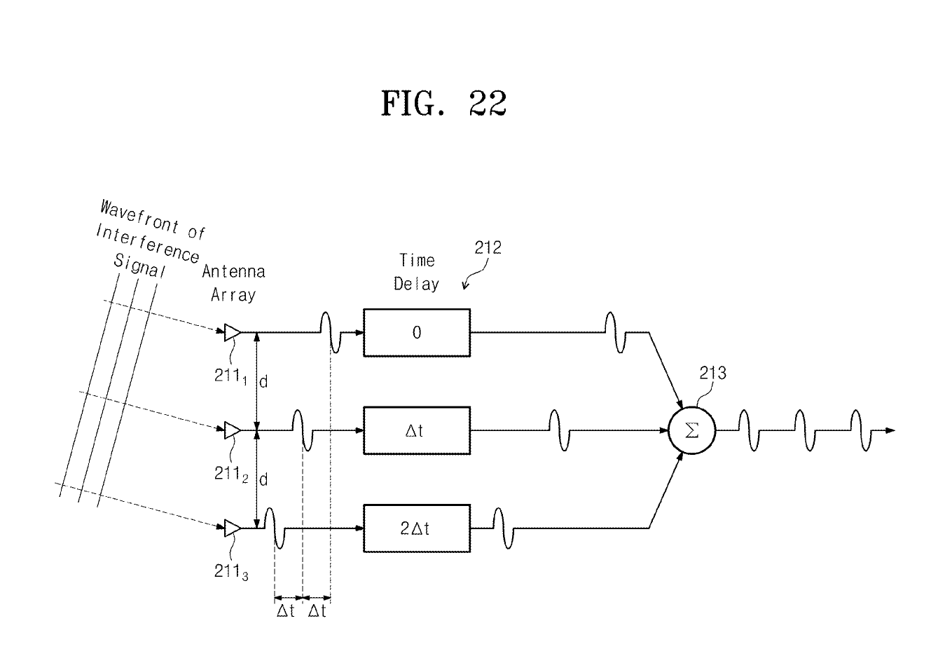

FIG. 22 is a diagram for describing an interference signal receiving method of a receiver 210, according to another exemplary embodiment of the inventive concept.

Unlike a signal transmitted through a straight line, a signal reflected and received through a multi-path may lower measurement accuracy about a bio signal. According to an exemplary embodiment of the inventive concept, a receiver 210 may exclude an interference signal transmitted through a multi-path, not a predetermined straight line path, in measuring a bio signal.

Referring to FIG. 22, interference signals received through antennas may be delayed by delays, applied at a transmission stage, that is, 0, Δt, and 2Δt, by a delay unit 212. Unlike signals transmitted through straight line paths illustrated in FIG. 21, phase differences between pulse signals corresponding to interference signals may become greater by the delay unit 212.

Accordingly, a pulse signal added by an adder 213 may be formed of a plurality of pulses having small amplitude without constructive interference as illustrated in FIG. 21.

According to an exemplary embodiment of the inventive concept, a processing unit 220 may select a pulse signal, having amplitude greater than a predetermined threshold value, from among pulse signals from the receiver 210 and may measure a bio signal of a to-be-measured person based on the selected pulse signal. In this case, the processing unit 220 may scale down a pulse signal of which the amplitude is amplified due to the constructive interference, so as to have an amplitude level of a pulse signal before constructive interference.

According to an exemplary embodiment of the inventive concept, the transmitter 100 and the receiver 210 may be synchronized to share a clock signal. For example, the transmitter 100 may transmit information about a clock signal used in the transmitter 100 to the receiver 210 to allow the transmitter 100 and the receiver 210 to share a clock signal. This may mean that the transmitter 100 and the receiver 210 are synchronized. The information about the clock signal may be transferred through a cable, but in some embodiments, it may be wirelessly transmitted.

According to an exemplary embodiment of the inventive concept, a vehicle may be equipped with the user monitoring system 10 to monitor passengers of the vehicle.

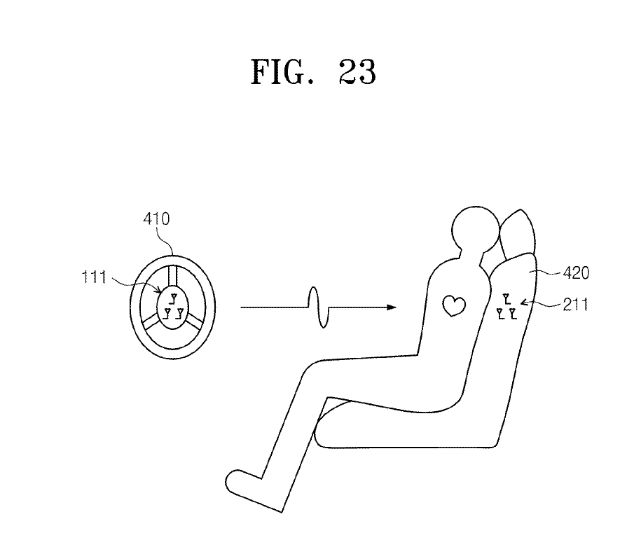

FIG. 23 is a diagram schematically illustrating arrangements of transmitter antennas 111 and receiver antennas 211 in a vehicle according to an exemplary embodiment of the inventive concept.

In the case where a vehicle is equipped with a user monitoring system 10 to monitor a driver of the vehicle, antennas 1111, 1112, and 1113 included in a transmitter 100 and antennas 2111, 2112, and 2113 included in a receiver 210 may be arranged on a straight line passing through a heart of the driver to transmit and receive pulse signals.

For example, as illustrated in FIG. 23, the transmitter antennas 111 may be mounted at a steering wheel 410 of the vehicle, and the receiver antennas 211 may be mounted at a backrest of a driver's seat.

Since a driver maintains a fixed posture in which a chest of the driver faces the steering wheel 410 after sitting in the driver's seat, the transmitter antennas 111 and the receiver antennas 211 may be arranged to face each other with a measuring part (e.g., a heart) interposed therebetween. Accordingly, a pulse signal may pass through the measuring part.

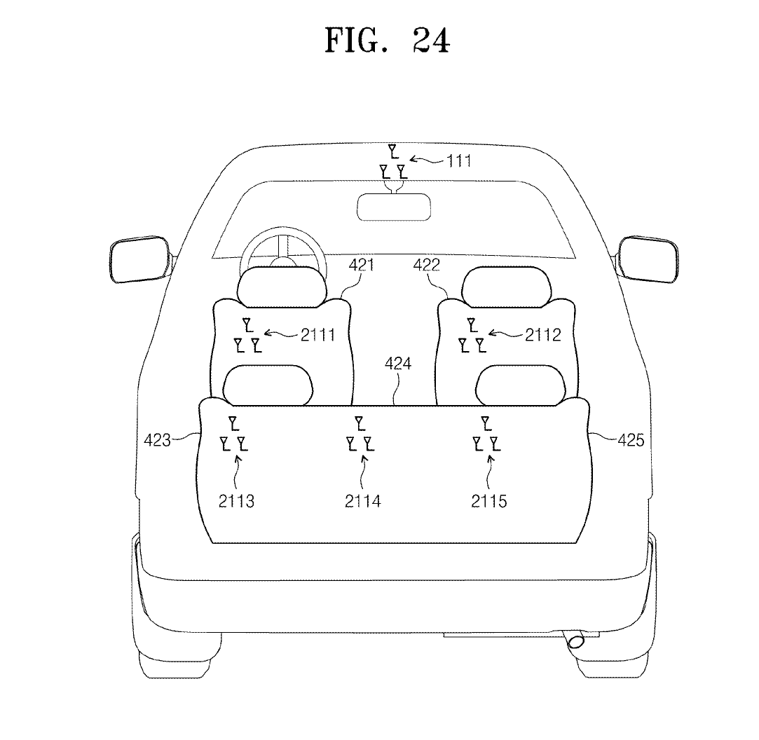

FIG. 24 is a diagram schematically illustrating a state where transmitter antennas 111 and receiver antennas 2111 to 2115 are disposed in a vehicle, according to another exemplary embodiment of the inventive concept.

According to another exemplary embodiment of the inventive concept, a user monitoring system 10 may monitor passengers as well as a driver. In this case, the user monitoring system 10 may include a transmitter 100 and a receiver 210 provided every passenger to measure bio signals of the passengers. In exemplary embodiments, it may be possible to measure bio signals of passengers using one transmitter 100.

For example, as illustrated in FIG. 24, transmitter antennas 111 may be mounted at a front, top side of the interior of a vehicle, and receiver antennas 2111 to 2115 may be mounted at backrests 421 to 424 of seats of the vehicle, respectively.

As such, in the case where the transmitter antennas 111 and the receiver antennas 2111 to 2115 are arranged in a vehicle, the transmitter antennas 111 may be disposed to face the receiver antennas 2111 to 2115 with measuring parts of all passengers interposed therebetween.

According to an exemplary embodiment of the inventive concept, a plurality of antennas 1111, 1112, and 1113 included in the transmitter 100 or a plurality of antennas 2111, 2112, and 2113 included in the receiver 219 may be located on the same plane.

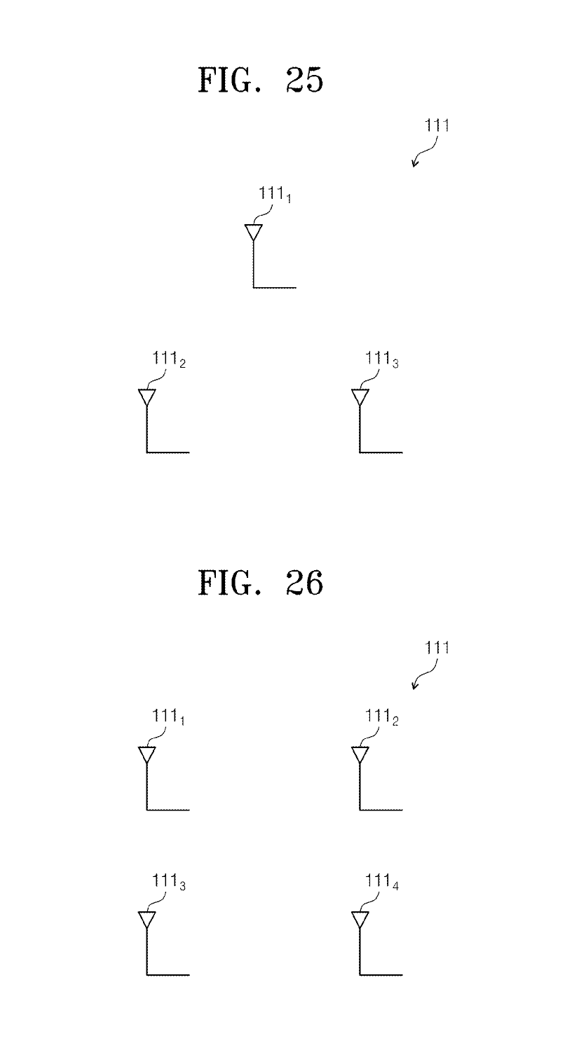

FIGS. 25 to 27 are diagrams schematically illustrating arrangement of antennas 111 according to exemplary embodiments of the inventive concept.

Antennas 111 may be located on one plane such that a polygon is formed when piecing the antennas 111 together.

For example, referring to FIG. 25, the antennas 111 may include three antennas 1111, 1112, and 1113, and the antennas 1111, 1112, and 1113 may be disposed on one plane to form a triangular shape.

As another example, referring to FIG. 26, the antennas 111 may include four antennas 1111, 1112, 1113, and 1114 and the antennas 1111, 1112, 1113, 1114 may be disposed on one plane to form a tetragonal shape.

The number of antennas included in the transmitter 100 or the number of antennas included in the receiver 210 may increase over 5 or more, not limited to 3 or 4.

Furthermore, the plurality of antennas 111 may further include an antenna, placed at the center of the polygon, as well as antennas corresponding to apexes of a polygon.

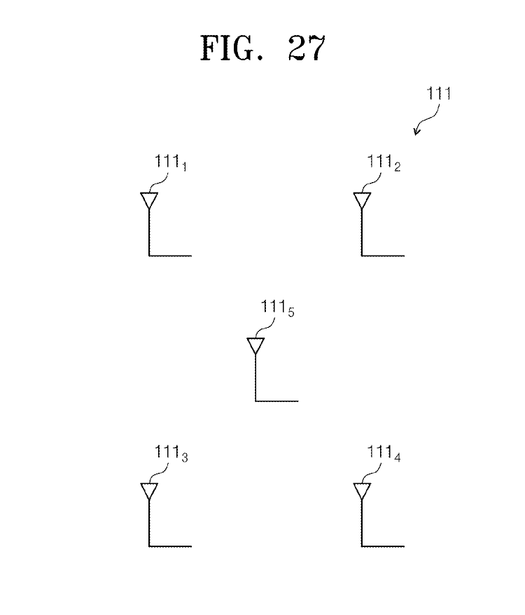

For example, referring to FIG. 27, the antennas 111 may further include an antenna 1115, placed at the center of a tetragon, as well as antennas 1111, 1112, 1113, and 1114 corresponding to apexes of the tetragon.

The above-described arrangement of antennas may be applicable to antennas included in the receiver 210 as well as antennas included in the transmitter 100. That is, even though the transmitter 100 is mounted at a point in a vehicle and receivers 210 are mounted at backrests of all seats, the transmitter 100 may transmit pulse signals toward a receiver 210 by appropriately adjusting a delay of a pulse signal to be transmitted through each antenna.

Returning to FIG. 1, a user monitoring system 10 may further include an input device 300. The input device 300 may be a device to allow a user to enter information associated with an operation of the user monitoring system 10 and may include, for example, a touch screen, a keypad, and the like.

According to an exemplary embodiment of the inventive concept, the input device 300 may input information associated with a seat where a passenger (or a person to be measured) of a vehicle sits. In this case, a transmitter 100 may transmit pulse signals toward receiver antennas mounted at a backrest of the seat where the passenger sits.

For example, in the case where persons sit at a driver's seat and a rear, right seat of five seats illustrated in FIG. 24, the input device 300 may receive an input for selecting the driver's seat and the rear, right seat as seats where persons to be measured sit, from a user (e.g., a driver).

Accordingly, the transmitter 100 may not transmit pulse signals to all receiver antennas 2111 to 2115 mounted at a vehicle, but it may transmit the pulse signals to backrests 421 and 425 of seats selected according to the input, thereby preventing unnecessary transmission and reception and processing of pulse signals.

An embodiment of the inventive concept is exemplified as the input device 300 receives information associated with a seat where a to-be-measured person sits, from a user. In some embodiments, the input device 300 may receive whether a to-be-measured person boards, from sensors respectively mounted at seats.

For example, sensors may be mounted at seat belt buckles of all seats. In the case where a seat belt is joined with the buckle, a sensor mounted at the buckle may notify the input device 300 that a to-be-measured person sits in a seat corresponding to the buckle.

According to an exemplary embodiment of the inventive concept, in the case where a seat where a to-be-measured person sits is in plurality, the transmitter 100 may transmit pulse signals toward a plurality of antennas 2111 and 2115 mounted at backrests of the seats corresponding to persons to be measured, in a time division method. That is, the transmitter 100 may transmit pulse signals to a plurality of antennas 2111 and 2115 at different times.

In the case where the user monitoring system measures a heartbeat of a to-be-measured person, a heartbeat period may generally be hundreds milliseconds or several seconds. For this reason, if an impulse corresponding to a nanosecond unit is used to measure a heartbeat, time slots may be allotted by the number of passengers to measure heartbeats of persons to be measured.

According to another exemplary embodiment of the inventive concept, the user monitoring system 10 may adjust a transmission direction of a pulse signal according to a physique of a person to be measured.

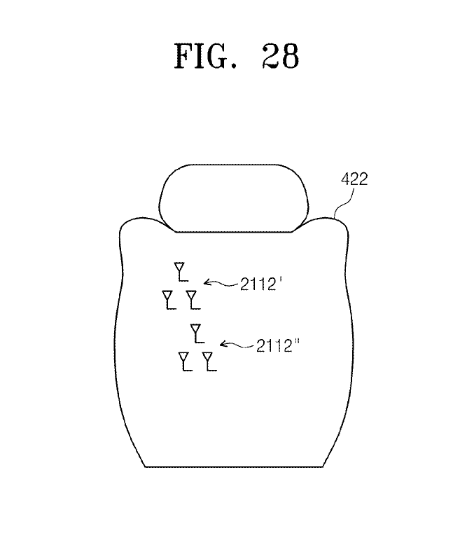

FIG. 28 is a diagram schematically illustrating arrangement of receiver antennas 2112′ and 2112″ in a vehicle, according to still another exemplary embodiment of the inventive concept.

As illustrated in FIG. 28, according to still another exemplary embodiment of the inventive concept, a receiver 210 may include a first antenna group 2112′ mounted at a first portion of a backrest 422 of a seat and a second antenna group 2112″ mounted at a second portion of the backrest 422.

An input device 300 may receive information associated with a physique of a to-be-measured person that boards a vehicle. A transmitter 100 may transmit pulse signals toward an antenna group, corresponding to the physique of the -be-measured person, from among the first and second antenna groups 2112′ and 2112″.

For example, the first antenna group 2112′ formed of a plurality of antennas may be mounted at a portion, corresponding to a heart of an adult, of a backrest 422, and the second antenna group 2112″ formed of a plurality of antennas may be mounted at a portion, corresponding to a heart of a child, of the backrest 422.

In the case where a user (e.g., a driver) inputs, through an input device 300, information atomic sequencer a physique of a to-be-measured person, for example, information corresponding to a child with respect to seats where the to-be-measured person sits, the transmitter 100 may transmit pulse signals toward the second antenna group 2112″ being an antenna group corresponding to a child, based on the input information.

In exemplary embodiments, a physique of a person to be measured may be classified as an adult or a child, as an age, as sex, or as stature. That is, as information associated with a physique of a person to be measured, a user may not select one of an adult and a child, but may select one of male or female or input an age or stature of the person to be measured.

According to exemplary embodiments of the inventive concept, a bio signal may be measured using a pulse signal transmitted through a straight line path passing through a measuring part of a to-be-measured person, and interference due to reflected signals transmitted through a multi-path may be excluded. Accordingly, it may be possible to improve accuracy of measurement.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A user monitoring system comprising:
a transmitter configured to transmit a pulse signal through at least one antenna at different times; and
a biosignal measuring device comprising a receiver and a processing unit,
wherein the receiver receives and restores the pulse signal that has passed through a subject through at least one antenna, and
wherein the processing unit processes the pulse signal to analyze at least one of a bandwidth, a center frequency and an amplitude of the pulse signal and measures a biosignal of the subject on the basis of a result of analysis.

2. The user monitoring system of claim 1, wherein a pulse of the pulse signal is repeated at a preset pulse repetition period.

3. The user monitoring system of claim 1, wherein the transmitter comprises a delay unit configured to provide, to the at least one antenna of the transmitter, a plurality of pulse signals obtained by different delays to the pulse signal.

4. The user monitoring system of claim 1, wherein the receiver comprises:
a delay unit configured to apply different delays to a plurality of pulse signals received by the at least one antenna of the receiver; and
an adder configured to add the plurality of pulse signals output by the delay unit.

5. The user monitoring system of claim 1, wherein the transmitter and the receiver are synchronized with each other to share a clock signal.

6. The user monitoring system of claim 1, wherein the user monitoring system is provided to a vehicle to monitor the subject in the vehicle.

7. The user monitoring system of claim 6, wherein the at least one antenna of the transmitter is installed in a steering wheel of the vehicle, and the at least one antenna of the receiver is installed in a back of a driver's seat of the vehicle.

8. The user monitoring system of claim 6, wherein the at least one antenna of the transmitter is installed at a front upper part in the vehicle, and the at least one antenna of the receiver is installed in a back of a seat of the vehicle.

9. The user monitoring system of claim 8, wherein a plurality of antennas are such arranged that the antennas are flush with each other and form a shape of a polygon.

10. The user monitoring system of claim 9, further comprising an antenna disposed at a center of the polygon.

11. The user monitoring system of claim 8, further comprising:
an input device configured to receive information on the seat of the vehicle on which the subject is seated,
wherein the transmitter transmits the pulse signal towards the at least one antenna installed in the back of the seat on which the subject is seated.

12. The user monitoring system of claim 11, wherein, in the case where the subject is seated on a plurality of seats, the transmitter transmits the pulse signal towards the at least one antenna installed in a back of each seat on which the subject is seated at different times.

13. The user monitoring system of claim 6, further comprising:
an input device configured to receive information on a physique of the subject in the vehicle,
wherein the receiver comprises a first antenna group installed in a first part of a back of a seat of the vehicle and a second antenna group installed in a second part of the back of the seat,
wherein the transmitter transmits the pulse signals towards one of the first and second antenna groups which corresponds to the physique of the subject.
